(19) United States
(12) Patent Application Publication
(10) Pub. No.: US 2024/0105321 A1
(43) Pub. Date: Mar. 28, 2024

(54) NON-TRANSITORY COMPUTER READABLE MEDIUM

(71) Applicant: FUJIFILM Corporation, Tokyo (JP)

(72) Inventors: Keiji TSUBOTA, Kanagawa (JP); Noriaki IDA, Tokyo (JP); Shozo IYAMA, Kanagawa (JP)

(73) Assignee: FUJIFILM Corporation, Tokyo (JP)

(21) Appl. No.: 18/472,274

(22) Filed: Sep. 22, 2023

(30) Foreign Application Priority Data
Sep. 27, 2022 (JP) 2022-153341

(51) Int. Cl. G16H 40/20; G16H 40/67

(52) U.S. Cl. CPC G16H 40/20; G16H 40/67

(57) ABSTRACT

A health checkup support program according to an aspect of the present invention causes a central control unit of a smart watch that acquires biological information of a wearer to realize a health checkup information detection function of detecting a scheduled implementation date and time of a health checkup of the wearer by accessing a health checkup information server that stores health checkup information related to the health checkup, an ineligible matter detection function of monitoring the biological information of the wearer obtained via the wearable device for a predetermined period until the scheduled implementation date and time of a health checkup, and detecting an ineligible matter that is ineligible for implementing the health checkup, and a health checkup ineligibility notification function of, in a case in which the ineligible matter is detected, notifying of the fact.

### CROSS-REFERENCE TO RELATED APPLICATIONS

This application claims priority under 35 U.S.C § 119(a) to Japanese Patent Application No. 2022-153341 filed on 27 Sep. 2022. The above application is hereby expressly incorporated by reference, in its entirety, into the present application.

### BACKGROUND OF THE INVENTION

### 1. Field of the Invention

The present invention relates to a non-transitory computer readable medium for storing a computer-executable program for causing a computer of a wearable device that acquires biological information of a wearer to realize a predetermined function.

### 2. Description of the Related Art

As the wearable device that acquires the biological information of the wearer, for example, a smart watch is known. Since the smart watch can acquire a body temperature, a heart rate, a blood pressure, a blood glucose level, and the like as the biological information of the wearer, it is effective for daily health management (see JP2022-003554A (corresponding to US2018/329584A1) and JP2022-037048A (corresponding to US2019/103007A1) below).

### SUMMARY OF THE INVENTION

As in JP2022-003554A and JP2022-037048A, measuring (collecting) the biological information with the wearable device is effective for daily health management, but in order to lead a healthier life, it is preferable to have a regular checkup by a doctor or the like, such as a health checkup.

However, in the health checkup, there is a rule such as not eating or drinking after 9 o'clock on the day before the health checkup, and in a case in which such a rule is not observed, there is a problem that an appropriate result cannot be obtained. Such a problem is expected to become more pronounced in countries other than Japan, where employers are not obliged to have their employees undergo the health checkup.

The present invention has been made in view of the above background, and an object of the present invention is to provide a non-transitory computer readable medium for storing a computer-executable program for enabling an appropriate health checkup not only in Japan, which is familiar with the health checkup, but also in other countries, which are less familiar with the health checkup.

In order to solve the problem, according to the present invention, there is provided a non-transitory computer readable medium for storing a computer-executable program for causing a computer of a wearable device that acquires biological information of a wearer to realize a predetermined function comprising: a health checkup information detection function of detecting a scheduled implementation date and time of a health checkup of the wearer by accessing a health checkup information storage unit that stores health checkup information related to the health checkup; an ineligible matter detection function of monitoring the biological information of the wearer obtained via the wearable device for a predetermined period until the scheduled implementation date and time of a health checkup detected by the health checkup information detection function, and detecting an ineligible matter that is ineligible for implementing the health checkup; and a health checkup ineligibility notification function of, in a case in which the ineligible matter is detected by the ineligible matter detection function, notifying of the fact.

It is preferable that the health checkup information storage unit stores a detection criterion for the ineligible matter as the health checkup information, and the ineligible matter is detected using the detection criterion.

It is preferable that the predetermined function includes a detection criterion notification function of notifying of the detection criterion.

It is preferable that the health checkup information storage unit stores a diagnostic item in the health checkup of the wearer as the health checkup information, and the detection criterion is determined for each diagnostic item.

It is preferable that the ineligible matter detection function is a function of detecting that the biological information of the wearer is beyond a predetermined eligibility range, as the ineligible matter.

It is preferable that the predetermined function includes a behavior detection function of detecting a behavior of the wearer, and the ineligible matter detection function is a function of detecting an ineligible behavior, which is ineligible for implementing of the health checkup, among the behaviors detected by the behavior detection function.

It is preferable that the ineligible behavior is eating or drinking outside a permissible period.

It is preferable that the ineligible behavior is implementation of exercise beyond a permissible range.

It is preferable that the ineligible behavior is ingestion or administration of a medicine beyond a permissible range.

It is preferable that the wearable device acquires position information of the wearer, and the ineligible matter detection function is a function of detecting the ineligible behavior by using the position information of the wearer obtained via the wearable device in addition to the biological information of the wearer.

It is preferable that the predetermined function includes a health checkup date and time change function of changing the scheduled implementation date and time of a health checkup stored in the health checkup information storage unit.

It is preferable that the health checkup date and time change function is enabled in a case in which the ineligible matter is detected.

According to the present invention, it is possible to perform an appropriate health checkup according to a rule.

### BRIEF DESCRIPTION OF THE DRAWINGS

FIG. 1 is a schematic diagram showing a configuration of a health checkup support system.

FIG. 2 is an explanatory diagram showing contents of health checkup schedule data.

FIG. 3 is an explanatory diagram showing contents of precautionary data.

FIG. 4 is a block diagram showing a schematic configuration of a smart watch.

FIG. 5 is a flowchart showing a flow of detection and notification of an ineligible matter.

FIG. 6 is a block diagram showing a schematic configuration of a smart watch.

FIG. 7 is a flowchart showing a flow of notification of a detection criterion for an ineligible matter.

FIG. 8 is a block diagram showing a schematic configuration of a smart watch.

FIG. 9 is a flowchart showing a flow of change of a scheduled implementation date and time of a health checkup.

### DESCRIPTION OF THE PREFERRED EMBODIMENTS

### First Embodiment

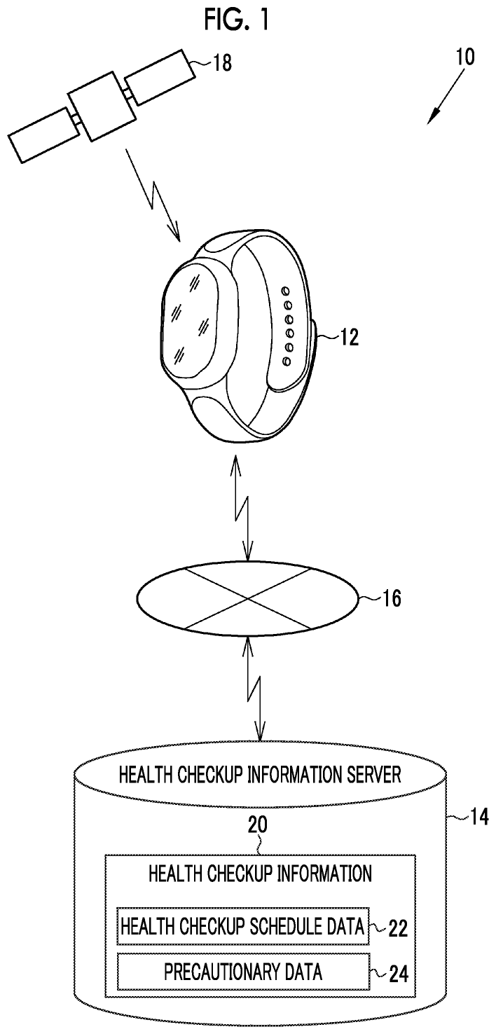

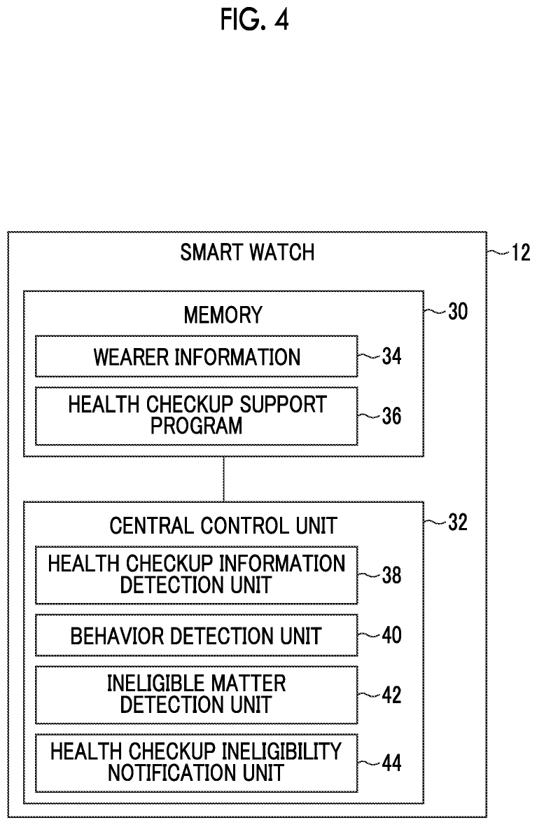

In FIG. 1, a health checkup support system 10 is for supporting an appropriate execution of a health checkup. The health checkup support system 10 comprises a smart watch 12 (wearable device) in which a health checkup support program 36 (see FIG. 4) according to an embodiment of the present invention is installed, and a health checkup information server 14 (health checkup information storage unit), and these are communicably connected to each other via a network 16 such as the Internet.

The smart watch 12 is a watch-type wearable device to be worn on a wrist. The smart watch 12 has a position information acquisition function of receiving radio waves from a global positioning system (GPS) satellite 18 and detecting a current position.

In addition, the smart watch 12 comprises dedicated sensors for acquiring biological information, such as a blood glucose level sensor and a blood pressure sensor, as well as an image sensor, a temperature sensor, a pressure sensor, a vibration sensor, and a magnetic sensor, and has a biological information acquisition function of acquiring biological information (body temperature, pulse rate, blood pressure, blood glucose level, or the like) of the wearer by using information from these sensors.

An example of measuring a blood glucose level with a wearable device such as a smart watch is described at Address 1 and Address 2 below, and an example of measuring a blood pressure is described at Address 3 below.
Address 1
   Medical DX, “Apple Watch also targets needle-free blood glucose level measurement, what's the research result?—a new relationship between a smart watch and diabetes treatment”, [online], Mar. 29, 2022, Medical DX editorial department, [Searched on Sep. 20, 2022], Internet
   <URL: https://medicaldx-jp.com/news/330>
Address 2
   Akira Minamikawa, “Blood glucose measurement without blood sampling, with a smart watch”, [online], Sep. 24, 2021, Nihon Keizai Shimbun, [Searched on Sep. 20, 2022], Internet
   <URL: https://www.nikkei.com/article/DGXZQOUC08BUK0Y1A900C2000000/>
Address 3
   Men's hideout digital editorial department, “Two mechanisms to “measure” a blood pressure with a smart watch”, [online], Aug. 31, 2022, Men's hideout digital, [Searched on Sep. 20, 2022], Internet
   <URL: https://otokonokakurega.com/learn/ippin/16656/#%E3%82%B9%E3%83%9E%E3%83%BC %E3%83%88%E3%82%A6%E3%82%A9%E3%83%83%E3%83%81%E3%81%A7%E8%A 1%80%E5%9C%A7%E3%82%92-%E6%B8%AC%E5%AE%9A-%E3%81%99%E3%82%8B2%E3%81%A4%E3%81%AE%E4%BB%95%E7%B5%84%E3%81%BF>

In the present embodiment, an example of applying the present invention to a smart watch as a wearable device is described, but the present invention may be applied to a wearable device other than the smart watch.

The health checkup information 20 is stored in the health checkup information server 14. The health checkup information is information about the health checkup, and includes health checkup schedule data 22 related to an implementation schedule of the health checkup, and precautionary data 24 indicating precautions (detection criterion for ineligible matter) for implementing the health checkup.

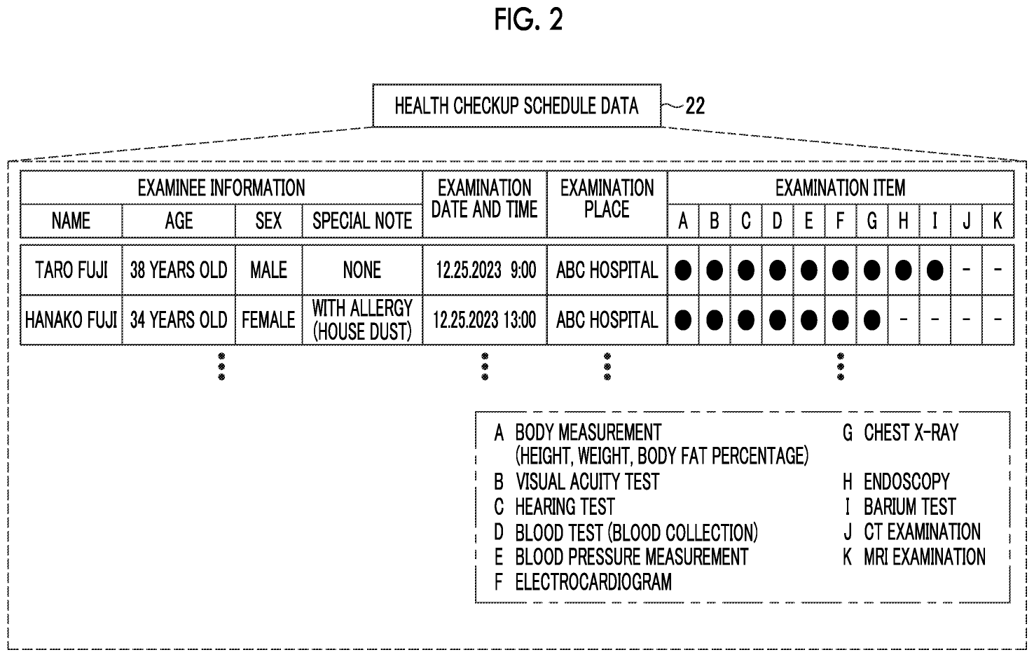

As shown in FIG. 2, the health checkup schedule data 22 includes examinee information, an examination date and time, an examination place, and an examination item. The examinee information is information about an examinee of the health checkup, and is information indicating a name, an age, a sex, and special notes (previous illness or chronic illness, injuries and diseases currently being treated, medicines prescribed and administered, and the like) of the examinee. The examination date and time is information indicating a date and time when the health checkup is scheduled to be implemented (scheduled implementation date and time of a health checkup). The scheduled health checkup place is information indicating a place where the health checkup is scheduled to be implemented. The examination item is information indicating a type of diagnosis (diagnostic item) to be implemented in the health checkup.

FIG. 2 shows an example in which information of two persons, a name “Taro Fuji” and a name “Hanako Fuji”, is stored as the health checkup schedule data 22. Then, the name “Taro Fuji” has the contents of an age “38 years old”, a sex “male”, special notes “none”, an examination date and time “9:00 AM, Dec. 25, 2023”, an examination place “ABC hospital”, and implementation (examination) of a health checkup for diagnostic items of “A to I” among diagnostic items of A to K. In addition, the name “Hanako Fuji” has the contents of an age “34 years old”, a sex “female”, special notes “with allergy (house dust)”, an examination date and time “1:00 PM, Dec. 25, 2023”, an examination place “ABC hospital”, and implementation (examination) of a health checkup for diagnostic items of “A to G” among diagnostic items of A to K.

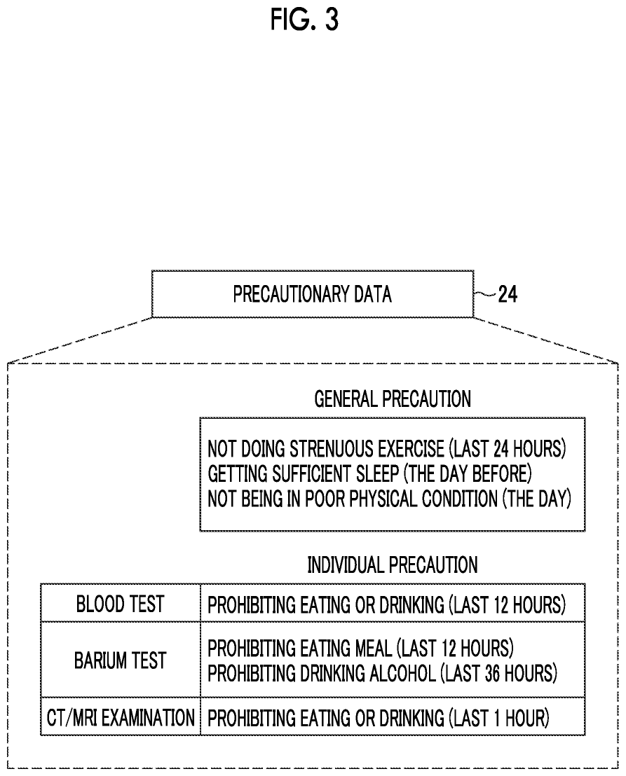

As shown in FIG. 3, the precautionary data 24 is information that serves as a detection criterion in a case of detecting ineligible matters (matters ineligible for health checkup), which will be described below. The precautionary data 24 includes information about general precautions related to all the diagnostic items and individual precautions for each diagnostic item. The general precautions include, for example, prohibiting strenuous exercise for the last 24 hours, getting sufficient sleep the day before, and not being in poor physical condition on the day. The individual precautions include prohibiting eating or drinking for the last 12 hours in a case of performing a blood test, prohibiting eating a meal for the last 12 hours and prohibiting drinking alcohol for the last 36 hours in a case of performing a barium test, and prohibiting eating or drinking for the last 1 hour in a case of performing a CT/MRI examination.

As shown in FIG. 4, the smart watch 12 is provided with a memory 30 and a central control unit 32. The memory 30 stores wearer information 34 about the wearer of the smart watch 12. The wearer information is information indicating that the wearer of the smart watch 12 is, for example, “Taro Fuji”.

In addition, the health checkup support program 36 is stored in the memory 30. The central control unit 32 functions as a health checkup information detection unit 38, a behavior detection unit 40, an ineligible matter detection unit 42, and a health checkup ineligibility notification unit 44 by operating the health checkup support program 36 stored in the memory 30.

The health checkup information detection unit 38 causes a computer (central control unit 32) of the smart watch 12 to realize a health checkup information detection function. The health checkup information detection unit 38 accesses the health checkup information server 14, and searches for an examinee (person scheduled to be examined) that matches the wearer information 34. Then, the scheduled implementation date and time of a health checkup of the searched examinee, that is, the wearer of the smart watch 12 is detected.

The behavior detection unit 40 causes a computer (central control unit 32) of the smart watch 12 to realize a behavior detection function. The behavior detection unit 40 monitors the biological information of the wearer obtained via the smart watch 12 for a predetermined period until the scheduled implementation date and time of a health checkup (for example, a period from 3 days before the scheduled implementation date and time of a health checkup to the scheduled implementation date and time of a health checkup) of the wearer of the smart watch 12, and detects a behavior that may violate the precautions (hereinafter, cautionary behavior) stored in the precautionary data 24 as the precautions for the health checkup. The cautionary behavior is, for example, strenuous exercise, or eating or drinking, and, in a case in which the wearer performs the cautionary behavior, the behavior detection unit 40 detects this based on a change in the biological information of the wearer or the like.

Specifically, in a case in which the heart rate exceeds a preset reference value (for example, 100 beats/minute) or in a case in which the heart rate is equal to or higher than a normal rate (for example, an average value since a start of the monitoring) by a predetermined percentage (for example, 30%), it is assumed that the wearer has exercised strenuously, and this is detected as a cautionary behavior. In addition, in a case in which the blood glucose level exceeds a preset reference value (for example, 120 mg/dL) or in a case in which the blood glucose level is equal to or higher than a normal level (for example, an average value since a start of the monitoring) by a predetermined percentage (for example, 30%), it is assumed that the wearer has eaten a meal, and this is detected as a cautionary behavior. In addition, the cautionary behavior may be detected by using the position information (change of the position information) of the wearer in addition to the biological information of the wearer.

The ineligible matter detection unit 42 causes a computer (central control unit 32) of the smart watch 12 to realize an ineligible matter detection function. The ineligible matter detection unit 42 detects ineligible matter. The ineligible matter is a matter that violates the precautions stored in the precautionary data 24 as the precautions for the health checkup.

The ineligible matter detection unit 42 monitors the biological information of the wearer obtained via the smart watch 12 for a predetermined period until the scheduled implementation date and time of a health checkup of the wearer of the smart watch 12 as with the behavior detection unit 40, and, in a case in which the biological information is beyond a predetermined eligibility range set in advance, the ineligible matter detection unit 42 regards the physical condition of the wearer as being abnormal, and detects this as an ineligible matter. In this case, for example, being in poor physical condition (high body temperature (high fever), unstable heartbeat, or the like) despite the day of the health checkup is detected as an ineligible matter.

In addition, in a case in which the behavior detection unit 40 detects the cautionary behavior, the ineligible matter detection unit 42 determines whether or not the cautionary behavior corresponds to the ineligible matter. In a case in which the cautionary behavior corresponds to the ineligible matter, the behavior detection unit 40 detects this cautionary behavior as an ineligible matter. In this case, for example, strenuous exercise within 24 hours of the scheduled implementation date and time of a health checkup, eating a meal within 12 hours of the scheduled implementation date and time of a health checkup despite having a blood test in the health checkup, or the like is detected as an ineligible matter.

The health checkup ineligibility notification unit 44 causes a computer (central control unit 32) of the smart watch 12 to realize a health checkup ineligibility notification function. In a case in which an ineligible matter is detected, the health checkup ineligibility notification unit 44 notifies the wearer of the detection of the ineligible matter by, for example, displaying a message on a display unit of the smart watch 12 or reproducing a message from a speaker of the smart watch 12.

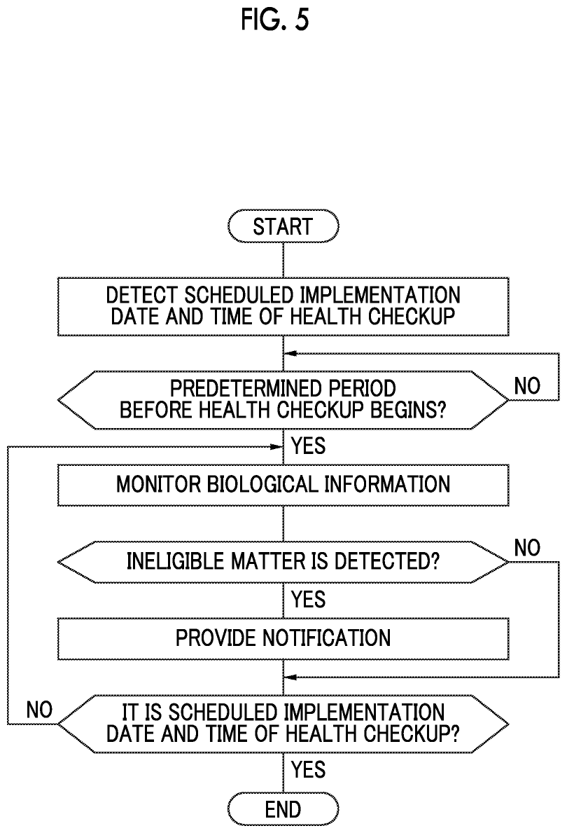

Hereinafter, a processing flow of the health checkup support program 36 will be described with reference to FIG. 5. As shown in FIG. 5, in a case in which the health checkup support program 36 is executed, the scheduled implementation date and time of a health checkup of the wearer of the smart watch 12 is detected, and the monitoring of the biological information of the wearer for a period from a predetermined period before the scheduled implementation date and time of a health checkup to the scheduled implementation date and time of a health checkup is started. Then, in a case in which an ineligible matter is detected as a result of the monitoring, a notification of the fact is given. As described above, according to the present invention, since the matters ineligible for implementing the health checkup are detected and notified, the wearer can take measures such as postponing the health checkup by explaining the circumstances to a doctor or the like at the time of the health checkup, and can have an appropriate health checkup.

Although the configuration of notifying the wearer of the smart watch 12 that the ineligible matter has been detected has been described as an example, the present invention is not limited thereto. A configuration of notifying a medical institution that performs a health checkup or a staff of the medical institution (a terminal of the medical institution or a terminal of the staff of the medical institution) that the ineligible matter has been detected may be adopted.

In addition, depending on a medicine, it may be possible to estimate the presence or absence of ingestion (administration) and the ingestion amount (dose) by monitoring the biological information. For example, it is possible to estimate ingestion of antihypertensive drugs such as amlodipine that lowers a blood pressure by monitoring a blood pressure level over time, and estimate ingestion of biguanides and sulfonylureas that lower a blood glucose level by monitoring a blood glucose level over time. Furthermore, depending on the diagnostic item of the health checkup, the ingestion (administration) or the ingestion amount (dose) of a specific medicine may be restricted. In a case in which the diagnosis of such an item is implemented in a health checkup, the presence or absence of ingestion and the ingestion amount of a specific medicine may be detected based on the biological information, and, in a case in which the detected matter violates the precautions, this may be detected as an ineligible matter.

A medicine that lowers a blood pressure is described at Address 4 and Address 5 below, and a medicine that lowers a blood glucose level is described at Address 6 below.
Address 4
   Taisuke Hattori, “Explanation of Amlodipine (a drug for hypertension)”, [online], Oct. 18, 2021, Asklepios Clinic, [Searched on Sep. 20, 2022], Internet
   <URL: https://asklepios-clinic.jp/blog/2021/10/18/amlodipine/>
Address 5
   Heart Clinic Fujisawa Mutsuai, “How to Choose Antihypertensive Drugs 1 ARB, ACE, Ca Antagonist”, [online], Nov. 29, 2021, Heart Clinic Fujisawa Mutsuai, [Searched on Sep. 20, 2022], Internet
   <URL: https://heartmutsuai.clinic/blog/?p=208>
Address 6
   National Center for Global Health and Medicine Diabetes Information Center, “Internal drugs that lower a blood glucose level”, [online], Jan. 24, 2022, National Center for Global Health and Medicine Diabetes Information Center, [Searched on Sep. 20, 2022], Internet
   <URL: http://dmic.ncgm.go.jp/general/about-dm/100/020/02. html>

In the present embodiment, the example of storing the health checkup support program 36 in the memory 30 has been described, but the present invention is not limited thereto. The health checkup support program 36 may be stored in a storage unit (for example, the health checkup information server 14 or the like) provided separately from the smart watch 12 and capable of communicating with the smart watch 12. In this case, the smart watch 12 (central control unit 32) need only access the storage unit to operate (read out, execute) the health checkup support program.

In addition, in the present embodiment, the smart watch 12 (central control unit 32) operates the health checkup support program 36 to function as the health checkup information detection unit 38, the behavior detection unit 40, the ineligible matter detection unit 42, and the health checkup ineligibility notification unit 44, but the present invention is not limited thereto. A configuration may be adopted in which a processor device capable of communicating with the smart watch 12 is provided separately from the smart watch 12, and this processor device operates the health checkup support program 36, and functions as the health checkup information detection unit 38, the behavior detection unit 40, the ineligible matter detection unit 42, and the health checkup ineligibility notification unit 44.

### Second Embodiment

While the first embodiment is configured to, in a case in which an ineligible matter is detected, notify of the smart watch 12 of the fact, the second embodiment is configured to notify the wearer of the smart watch 12 of the detection criterion for the ineligible matter in advance, in addition to the notification of the first embodiment. In the description of the second and subsequent embodiments, the same configurations as those of the above-described embodiment are denoted by the same reference numerals, and the description thereof is omitted.

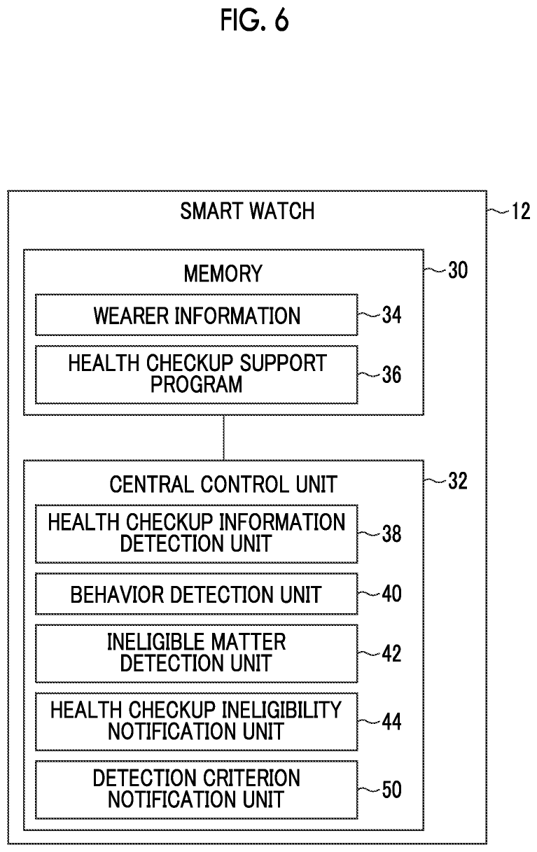

As shown in FIG. 6, in the second embodiment, by operating the health checkup support program 36 stored in the memory 30, the central control unit 32 functions as a detection criterion notification unit 50 in addition to the above-described units. The detection criterion notification unit 50 causes a computer (central control unit 32) of the smart watch 12 to realize a detection criterion notification function.

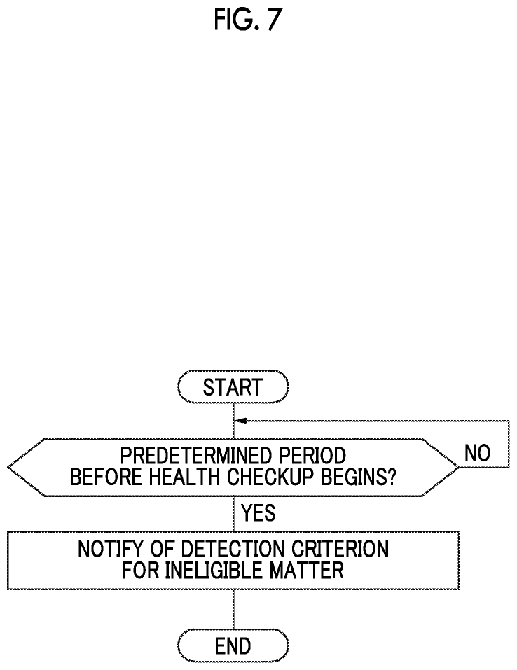

As shown in FIG. 7, when a predetermined period before the scheduled implementation date and time of a health checkup begins, the detection criterion notification unit 50 accesses the health checkup information server 14 to read out the precautionary data 24, that is, the detection criterion for the ineligible matter, and notifies the wearer of the smart watch 12 of the detection criterion by, for example, displaying a message on the display unit of the smart watch 12 or reproducing a message from the speaker of the smart watch 12. As described above, in the second embodiment, by notifying of the detection criterion for the ineligible matter, it becomes clear what precautions should be taken for the health checkup, and since it is possible to take action such that the ineligible matter is not detected, an appropriate health checkup can be performed.

### Third Embodiment

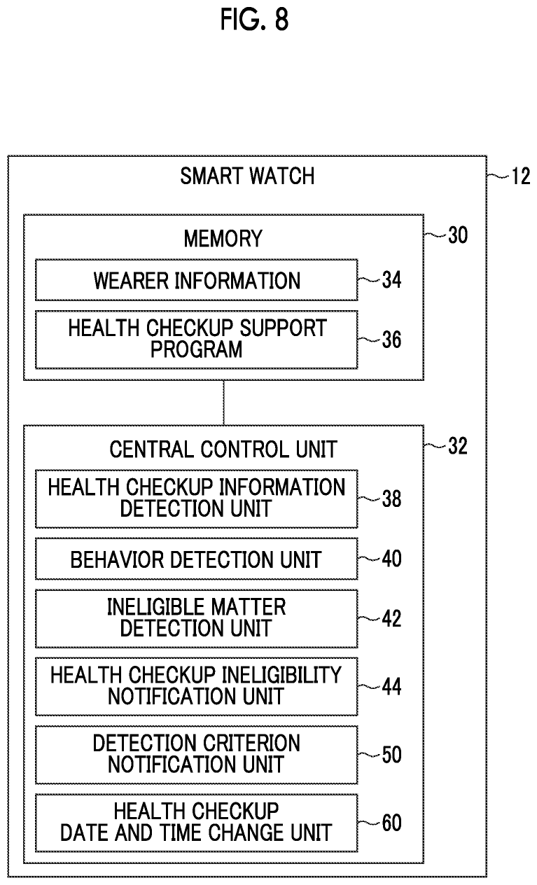

A third embodiment is configured to enable a change of the scheduled implementation date and time of a health checkup stored in the health checkup information server 14. As shown in FIG. 8, in the third embodiment, by operating the health checkup support program 36 stored in the memory 30, the central control unit 32 functions as a health checkup date and time change unit 60 in addition to the above-described units. The health checkup date and time change unit 60 causes a computer (central control unit 32) of the smart watch 12 to realize a health checkup date and time change function.

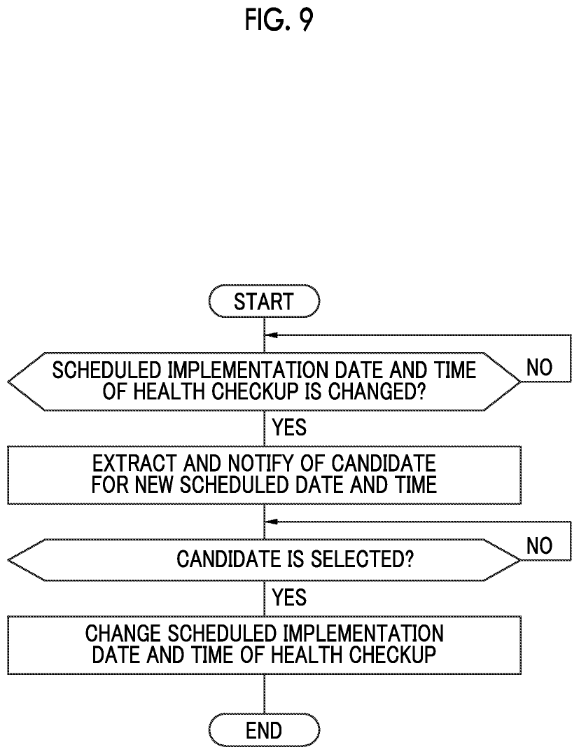

As shown in FIG. 9, in a case in which the wearer of the smart watch 12 selects to change the scheduled implementation date and time of a health checkup, the health checkup date and time change unit 60 starts a process of changing the scheduled implementation date and time of a health checkup. The scheduled implementation date and time of a health checkup can be changed at any timing by operating the smart watch 12. In addition, a configuration may be configured in which the scheduled implementation date and time of a health checkup can be changed by enabling the health checkup date and time change function in the case in which an ineligible matter is detected.

In a case in which the change of the scheduled implementation date and time of a health checkup is selected, the health checkup date and time change unit 60 accesses a reservation system of a medical institution that implements the health checkup or the like to extract candidates for a new scheduled date and time, notifies of the extracted candidates by displaying the extracted candidates on the display unit of the smart watch 12, and allows the wearer to select a candidate. Then, in a case in which a candidate is selected, a reservation for a current health checkup is canceled, and a reservation for a health checkup is made at the selected new scheduled date and time. In addition, the scheduled implementation date and time of a health checkup stored in the health checkup information server 14 is changed (updated) in accordance with the new reservation.

In the above embodiment, a hardware structure of a processing unit that executes various kinds of processing, such as the health checkup information detection unit 38, the behavior detection unit 40, the ineligible matter detection unit 42, the health checkup ineligibility notification unit 44, the detection criterion notification unit 50, and the health checkup date and time change unit 60, is various processors as shown below. The various processors include a central processing unit (CPU) that is a general-purpose processor that executes software (programs) to function as various processing units, a programmable logic device (PLD) that is a processor capable of changing a circuit configuration after manufacture, such as a field programmable gate array (FPGA), and an exclusive electric circuit that is a processor having a circuit configuration exclusively designed to execute various kinds of processing.

One processing unit may be configured of one of these various processors, or may be configured of a combination of two or more processors of the same type or different types (for example, a combination of a plurality of FPGAs or a combination of a CPU and an FPGA). In addition, a plurality of processing units may be configured of one processor. As an example in which the plurality of processing units are configured of one processor, first, as typified by computers such as a client or a server, one processor is configured of a combination of one or more CPUs and software, and this processor functions as the plurality of processing units. Second, as typified by a system on chip (SoC) or the like, a processor that realizes the functions of the entire system including the plurality of processing units by using one integrated circuit (IC) chip is used. As described above, the various processing units are configured using one or more of the various processors as a hardware structure.

Further, the hardware structure of these various processors is more specifically an electric circuit (circuitry) in a form in which circuit elements such as semiconductor elements are combined.

### EXPLANATION OF REFERENCES

10: health checkup support system
12: smart watch
14: health checkup information server (health checkup information storage unit)
16: network
18: GPS satellite
20: health checkup information
22: health checkup schedule data
24: precautionary data
30: memory
32: central control unit
34: wearer information
36: health checkup support program
38: health checkup information detection unit
40: behavior detection unit
42: ineligible matter detection unit
44: health checkup ineligibility notification unit
50: detection criterion notification unit
60: health checkup date and time change unit

What is claimed is:

1. A non-transitory computer readable medium for storing a computer-executable program for causing a computer to function as a wearable device that acquires biological information of a wearer, the computer-executable program causing the computer to execute:
a health checkup information detection function of detecting a scheduled implementation date and time of a health checkup of the wearer by accessing a health checkup information storage that stores health checkup information related to the health checkup;
an ineligible matter detection function of monitoring the biological information of the wearer obtained via the wearable device for a predetermined period until the scheduled implementation date and time of a health checkup detected by the health checkup information detection function, and detecting an ineligible matter that is ineligible for implementing the health checkup; and
a health checkup ineligibility notification function of, in a case in which the ineligible matter is detected by the ineligible matter detection function, notifying of the fact.

2. The non-transitory computer readable medium according to claim 1,
wherein the health checkup information storage stores a detection criterion for the ineligible matter as the health checkup information, and
the ineligible matter is detected using the detection criterion.

3. The non-transitory computer readable medium according to claim 2,
wherein the computer-executable program causes the computer to execute a detection criterion notification function of notifying of the detection criterion.

4. The non-transitory computer readable medium according to claim 3,
wherein the health checkup information storage stores a diagnostic item in the health checkup of the wearer as the health checkup information, and
the detection criterion is determined for each diagnostic item.

5. The non-transitory computer readable medium according to claim 1,
wherein the ineligible matter detection function is a function of detecting that the biological information of the wearer is beyond a predetermined eligibility range, as the ineligible matter.

6. The non-transitory computer readable medium according to claim 1,
wherein the computer-executable program causes the computer to execute a behavior detection function of detecting a behavior of the wearer, and
the ineligible matter detection function is a function of detecting an ineligible behavior, which is ineligible for implementing of the health checkup, among the behaviors detected by the behavior detection function.

7. The non-transitory computer readable medium according to claim 6,
wherein the ineligible behavior is eating or drinking outside a permissible period.

8. The non-transitory computer readable medium according to claim 6,
wherein the ineligible behavior is implementation of exercise beyond a permissible range.

9. The non-transitory computer readable medium according to claim 6,
wherein the ineligible behavior is ingestion or administration of a medicine beyond a permissible range.

10. The non-transitory computer readable medium according to claim 6,
wherein the wearable device acquires position information of the wearer, and
the ineligible matter detection function is a function of detecting the ineligible behavior by using the position information of the wearer obtained via the wearable device in addition to the biological information of the wearer.

11. The non-transitory computer readable medium according to claim 1,
wherein the computer-executable program causes the computer to execute a health checkup date and time change function of changing the scheduled implementation date and time of a health checkup stored in the health checkup information storage.

12. The non-transitory computer readable medium according to claim 11,
wherein the health checkup date and time change function is enabled in a case in which the ineligible matter is detected.
